(19) United States
(12) Patent Application Publication
(10) Pub. No.: US 2026/0000871 A1
(43) Pub. Date: Jan. 1, 2026

(54) Intravenous Catheterization Therapy Support Device

(71) Applicant: CROSSROAD TRAVEL NURSING LLC, Garden City, KS (US)

(72) Inventor: Beau Hovorka, Garden City, KS (US)

(73) Assignee: CROSSROAD TRAVEL NURSING LLC, Garden City, KS (US)

(21) Appl. No.: 19/237,246

(22) Filed: Jun. 13, 2025

Related U.S. Application Data
(60) Provisional application No. 63/664,276, filed on Jun. 26, 2024.

(51) Int. Cl. A61M 25/02

(52) U.S. Cl. CPC A61M 25/02; A61M 2025/0213; A61M 2025/0253; A61M 2205/0216; A61M 2205/0222; A61M 2209/088; A61M 2210/083

(57) ABSTRACT

The intravenous therapy support device has a securement and a cover structured to swivel. Two elongated and resilient pad members extend outwardly from the cover. Each pad member has a low-friction fabric cover layer, a support layer, and a comfort layer. The support layer has a higher density than that of the comfort layer. Pad members are spaced apart and aligned parallel to each other to create a central elongated opening that accommodates the intravenous catheter. The cover swivels to align the central elongated opening with a longitudinal axis of the intravenous catheter. Securement of the device on a patient stabilizes the patency of the intravenous catheter to prevent occlusion.

### CROSS REFERENCES

This application claims priority of U.S. Provisional Application No. 63/664,276 filed 26 Jun. 2024, which is incorporated herein by reference in its entirety.

### REFERENCE TO RESEARCH

Not Applicable.

### REFERENCE TO CDS

Not Applicable.

### FIELD OF THE INVENTION

The present disclosure relates to a medical device that applies to the body of a patient to provide patency of an intravenous catheter inserted into an intravenous catheterization therapy treatment site of the patient.

### BACKGROUND

One of the most utilized devices in medicine is the intravenous catheter. Intravenous therapy is frequently applied through insertion of intravenous catheters into patients that come into the hospital for ailments or injuries, such as sepsis, hypotension (low blood pressure), massive trauma, etc.

Approaches to securing and stabilizing intravenous catheters typically involve the use of adhesive tapes, straps, or other securing means which may cause discomfort or skin irritation to the patient. Challenges associated with securing and stabilizing intravenous catheters still remain.

### SUMMARY

In some aspects, the techniques described herein relate to a medical device comprising: (a) a securement; (b) a cover structured to swivel upon the securement; (c) a first pad member that is elongated and resilient extending outwardly away from the cover, the first pad member has: (i) a first fabric cover layer that may be a low-friction material structured to contact a patient's skin; (ii) a first support layer adjacent to the cover; (iii) a first comfort layer between the first fabric cover layer and the first support layer, wherein the first support layer has a greater density than the first comfort layer; (d) a second pad member that is elongated and resilient extending outwardly away from the cover, the second pad member has: (i) a second fabric cover layer that may be a low-friction material structured to contact a patient's skin; (ii) a second support layer adjacent to the cover; (iii) a second comfort layer between the second fabric cover layer and the second support layer, wherein the second support layer has a greater density than the second comfort layer; (e) wherein the first pad member and the second pad member are parallel and laterally spaced apart so as to define a central elongated opening adjacent to the cover and whereupon swiveling the cover upon the securement positions the central elongated opening to facilitate an intravenous catheter disposed below and between the first pad member and the second pad member; and (f) at least a portion of the first pad member and the second pad member being disposed directly below a portion of the securement when the securement is secured to a portion of a patient stabilizes patency of the intravenous catheter.

In some aspects, the securement is a band having: (a) an elbow pocket which may be a patch of greater flexibility material that is centrally located within the securement band and opposite the cover when the securement band is secured around the intravenous therapy catheterization treatment site of the patient.

In some aspects, the first pad member and the second pad member that are applied to the patient resist maximum flexion of a joint by between 5-15%.

In some aspects, the height of the first pad member is between ¼ inch to 1 inch.

In some aspects, the first pad member comprises: (a) a first longitudinal portion having a first longitudinal portion end height; (b) a second longitudinal portion having a second longitudinal portion end height; (c) a longitudinal center portion having a longitudinal center portion height; and (d) wherein the longitudinal center portion height is greater between the cover and the patient's skin than the first longitudinal portion end height and the second longitudinal portion end height.

In some aspects, the first pad member and the second pad member are structured to maintain the central elongated opening though bent along their respective longitudinal axes when a joint upon which the medical device is secured bends so as to prevent occlusion of an intravenous catheter disposed within the central elongated opening.

In some aspects, swiveling of the cover upon the securement is resisted with the securement secured to the portion of the patient.

In some aspects, the first support layer is a high-density foam and the first comfort layer is a low-density foam.

In some aspects, the medical device is secured to the patient without being fastened to the intravenous catheter.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### BRIEF DESCRIPTION OF DRAWINGS

Aspects are illustrated by way of example, and not by way of limitation, in the accompanying drawings, wherein:

FIG. 1 is a plan view of a first side of an example of the intravenous therapy support device.

FIG. 2 is a plan view of a second side of the example of the intravenous therapy support device.

FIG. 3 is an elevation view of a cross-section of a moveable support of the intravenous therapy support device.

FIG. 4 is a top perspective view of the intravenous therapy support device secured to a patient with the moveable support applied to the antecubital fossa vicinity.

### DETAILED DESCRIPTION

The intravenous therapy support device comprises a cover that swivels on a securement to position dual pad members extending outwardly from the cover. The dual pad members are elongated and laterally spaced apart to define a central elongated opening. Parallel placement of the dual pad members on either side of an inserted intravenous catheter positions the central elongated opening to accommodate the intravenous catheter. The cover swivels upon the securement to align the central elongated opening with a longitudinal axis of the intravenous catheter and dispose the intravenous catheter between the dual pad members. The resilient material of the dual pad members distributes pressure and stabilizes the patency of the intravenous catheter with the support device applied to the patient. Each pad member includes a cover layer of low-friction fabric structured to contact the patient's skin, a support layer adjacent the cover disposed below the securement, and a comfort layer disposed between the cover layer and the support layer. The support layer is of a higher density material than that of the comfort layer. A pocket of greater flexibility material than that of the securement may be disposed centrally within the securement. The pocket may be positioned opposite that of the cover to receive an apex of a joint of the patient with the support device applied to the patient. The joint is permitted to bend to approximately 75% to 95% of a maximum flexion with the dual pad members resisting the maximum flexion. The support device maintains the central elongated opening stabilizing the intravenous catheter along its length, beyond the insertion site, to prevent occlusion of the intravenous catheter.

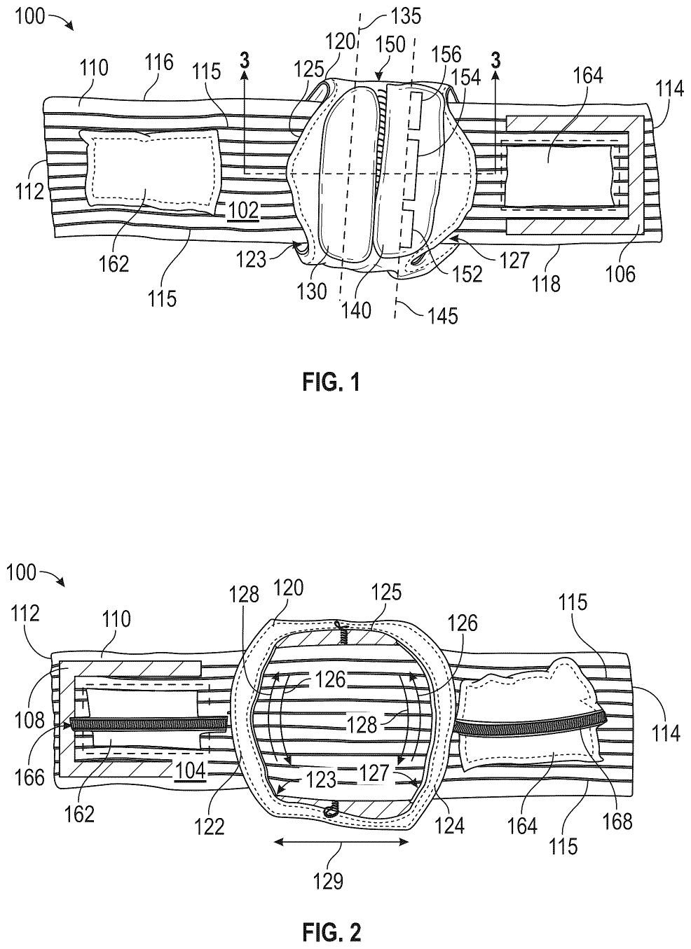

Now referring to the figures, a plan view of a first side 102 of an example intravenous therapy support device is depicted in FIG. 1. The intravenous therapy support device-hereinafter referred to as the “medical device 100”-comprises a securement 110. The securement 110 may be an elongated flat, thin band, strip, strap, or loop made of flexible or elastic materials that are soft and breathable, such as cotton, polyester, synthetic rubber, neoprene, nylon, spandex, polyurethane, or a combination of the materials, separated or blended, to ensure a clean and comfortable application of the medical device 100 against the patient's skin. The securement 110 may be notched with grooves 115 across the width and/or length of the material to allow for easy alignment on a portion of a patient and to provide a non-slip surface when applied against the patient to ensure the medical device 100 stays in alignment.

The securement 110 shown has a first end 112 and a second end 114. A first fastener attachment 106 may be disposed on a first attachment portion along the width and/or length of the securement 110 on the first side 102 at the second end 114. A second fastener attachment 108 may be disposed on a second attachment portion along the width and/or length of the securement 110 on a second side 104 at the first end 112. The fastener attachments may comprise strips of hook-and-loop fasteners (i.e. VELCRO®), buttons, adhesives, zippers, clasps, and/or other fastener solution types. The fastener attachments allow for easy application and removal of the medical device 100 to aid in protection and assessment of the intravenous catheterization therapy treatment site.

A first edge 116 and a second edge 118 extend along a length of the securement 110 between the first end 112 and the second end 114. The first edge 116 and second edge 118 may be adjacent and parallel to form a generally rectangular shape, with the medical device 100 having generally square corners at the intersections of the ends and edges of the securement 110. Medical device 100 may have other shapes, such as an elongated oval, curved arch, or be shaped with varied combinations. The first edge 116 and second edge 118 may be reinforced to prevent slipping and rolling of the securement 110.

First side 102 of securement 110 comprises a cover 125. Cover 125 may be made from a low-friction material, such as smooth, slick, breathable fabric, to facilitate contact with a patient's skin or intravenous dressing(s). Cover 125 may removably mount with the first side 102 of the securement 110. Alternatively, cover 125 may be mounted onto a base 120 that is structured move upon the securement 110 from the first end 112 towards the second end 114, or vice versa, as shown by movement arrow 129. Cover 125 may be structured to swivel upon the securement 110 with the base 120, as shown in FIG. 2 by movement arrows 126, 128. A swivel mechanism, such as a ball-and-socket joint, circular bearing connection, or hub and axle attachment may be utilized between cover 125 and securement 110 or base 120 for the swiveling action.

Swiveling may rotate the cover 125 and/or base 120 upon the securement 110 by a range of between 5° to 360°. The range of rotation of the cover 125 and/or base 120 upon the securement 110 may be further between 15° to 345°, between 30° to 330°, between 45° to 315°, between 60° to 300°, between 75° to 285°, between 90° to 270°, between 5° to 255°, between 15° to 240°, between 30° to 240°, between 45° to 225°, between 60° to 210°, between 75° to 195°, between 90° to 180°, between 5° to 165°, between 15° to 150°, between 30° to 135°, and between 45° to 120°. The range of rotation of the cover 125 and/or base 120 upon the securement 110 may be further defined by the degrees about which a central elongated opening 150 rotates from perpendicular (i.e. up to 45° clockwise/counterclockwise) to a longitudinal axis of the securement 110.

FIG. 2 depicts a plan view of the second side 104 of the medical device 100. As shown, base 120 may comprise a first base member 122 and a second base member 124 that are disposed on the second side 104. First base member 122 and second base member 124 may be elongated strips of material that span across a width of the securement 110 and are mounted to base 120 at points adjacent the first edge 116 and second edge 118. First base member 122 and second base member 124 may be made of the same low-friction material as the cover 125, or of a different material. First base member 122 and second base member 124 may be spaced apart from each other. First base member 122 and second base member 124 may extend perpendicularly straight across the width of the securement 110. First base member 122 and second base member 124 may extend across the width of the securement 110 as curved arches, convex to each other, being bowed outwards towards the first end 112 and the second end 114. Other shapes and configurations between the first base member 122 and second base member 124 are possible.

A first slot 123 may be disposed between the first base member 122 and the cover 125 of the base 120. A second slot 127 may be disposed between the first base member 122 and the cover 125 of the base 120. The securement 110 may be received into the first slot 123 and the second slot 127 so base 120 may slide upon the securement 110 from the first end 112 towards the second end 114, or vice versa, as shown by movement arrow 129. Having a base 120 that is slotted, with a circular shape may assist in the rotation of base 120 as shown by movement arrows 126, 128. Securement 110 utilized in conjunction with the base 120 that is movable upon the securement 110 leads to proper alignment and securement of the medical device 100 upon the patient to properly stabilize the intravenous catheter already inserted into the patient.

Referring back to FIG. 1, a first pad member 130 is shown disposed upon the base 120. The first pad member 130 extends outwardly away from the cover 125 of the base 120. The first pad member 130 may be securely fastened to cover 125. Material encasing the first pad member 130 may be integral with the cover 125 material or sewn to the cover 125 material. Alternatively, a first lower joining layer of medical-grade adhesive or VELCRO® may constitute a joining surface of the first pad member 130 for removable attachment of the first pad member 130 with the cover 125 or base 120. The first pad member 130 may be elongated, extending perpendicular to the length of the securement 110. The first pad member 130 may be made of a resilient material. The resilience of the material may be based on a factor of firmness or compression.

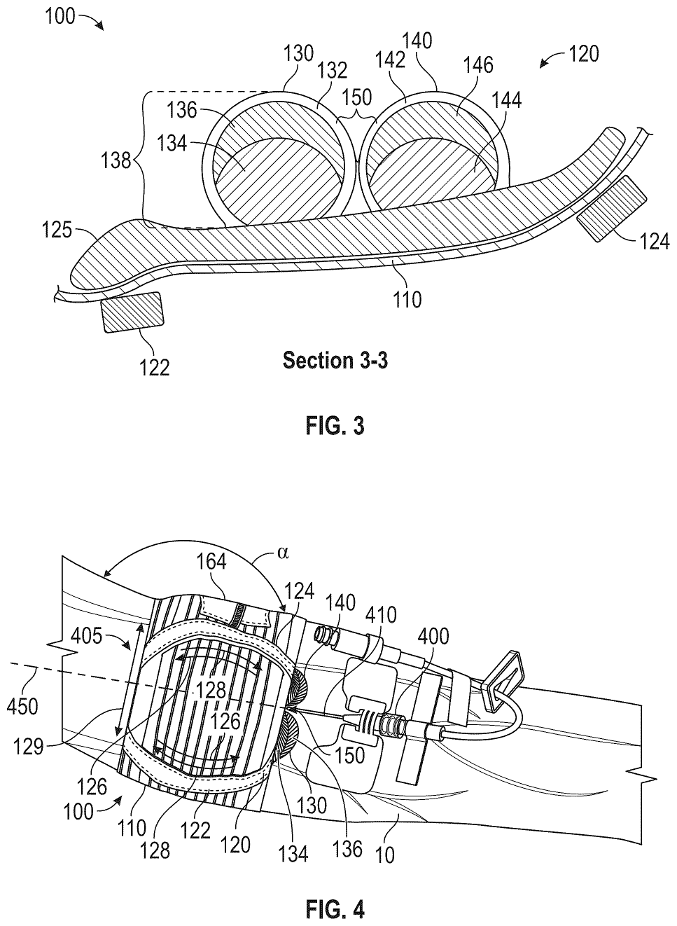

FIG. 3 depicts an elevation cross-sectional view of a Section 3-3 of the base 120 showing an example of the layering of materials within the first pad member 130 and a second pad member 140 of FIG. 1. First pad member 130 may be structured to contact a patient's skin with a first fabric cover layer 132. The first fabric cover layer 132 may be made of a low-friction material that is breathable. The low-friction material may provide an exterior surface that allows for low resistance during movement between the first pad member 130 and patient's skin. Comfort and support layers underlying the first fabric cover layer 132 are encased by the flexible and breathable material of the first fabric cover layer 132. The material of the first fabric cover layer 132 may be durable enough to hold the comfort and support layers in place at a fixed central area upon cover 125. The first fabric cover layer 132 may be comprised of materials such as cotton, polyester, synthetic rubber, neoprene, nylon, spandex, polyurethane, or a combination of the materials, separated or blended, to ensure a clean and comfortable application of the medical device 100 against the patient's skin.

The first pad member 130 comprises a first support layer 134 that underlies the first fabric cover layer 132 and is adjacent to cover 125. The first support layer 134 may be structured to support overlying layers of the first pad member 130. The first support layer 134 may be made of higher resilient, low compression set materials to provide support and resistance with pressure applied to the first pad member 130. The higher resilient material may be a high-density, less porous, less pliable, firm foam, sponge, rubber, or blended combination that may yield under higher degrees of compression. Foam utilized in the first support layer 134 as the higher resilient material may come from closed cell foam materials having high-density, such as foams from the family of polymers comprising polyethylene, polyurethane, silicone, and polyvinyl chloride (PVC). Sponge utilized in the first support layer 134 may comprise elastomers, like ethylene propylene diene terpolymer (EPDM) and Neoprene sponge having closed cells. Rubber utilized in the first support layer 134 may comprise elastomers that do not necessarily fit within the foam and sponge categories, like butyl rubber, cork, and cork rubber. A blended combination of these materials or other specialty foam materials having characteristics of low to non-sponginess may be utilized as the higher resilient material of the first support layer 134. The first support layer 134 has a greater density than a first comfort layer 136 of the first pad member 130.

The first comfort layer 136 may be disposed between the first fabric cover layer 132 and the first support layer 134. The first comfort layer 136 may be structured to provide cushioning between the patient's skin contacting the first fabric cover layer 132 and the higher resilient material of the first support layer 134. The first comfort layer 136 may be made of lower resilient, high compression set materials to prevent occlusion of intravenous vessels. The lower resilient material may be a low-density, more porous, more pliable, soft foam, sponge, rubber, or blended combination that may yield under lower degrees of compression. Foam utilized in the first comfort layer 136 as the lower resilient material may come from closed cell foam materials and open cell foam materials having low-density, such as foams from the family of polymers comprising urethane foam options, like ether or ester, microcellular urethane, memory foam, and reticulated filter foam. Sponge utilized in the first comfort layer 136 may comprise elastomers, like Neoprene sponge having open cells. Rubber utilized in the first comfort layer 136 may comprise elastomers that do not necessarily fit within the foam and sponge categories. A blended combination of these materials or other materials having greater sponginess may be utilized as the lower resilient material of the first comfort layer 136 to enhance patient comfort and reduce the risk of pressure and irritation at the intravenous catheterization therapy treatment site of the patient.

Referring back to FIG. 1, the second pad member 140 is shown disposed upon the base 120 and adjacent to the first pad member 130. The second pad member 140 extends outwardly away from cover 125 of the base 120. The second pad member 140 may be securely fastened to cover 125. Material encasing the second pad member 140 may be integral with the cover 125 material or sewn to the cover 125 material. Alternatively, a second lower joining layer of medical-grade adhesive or VELCRO® may constitute a joining surface of the second pad member 140 for removable attachment of the second pad member 140 with the cover 125 of the base 120. The second pad member 140 may be elongated, extending parallel with the first pad member 130 and perpendicular to the length of the securement 110. The second pad member 140 may be made of a resilient material. The resilience of the material may be based on a factor of firmness or compression.

FIG. 3 also shows an example of layering of materials within the second pad member 140. Second pad member 140 may be structured to contact a patient's skin with a second fabric cover layer 142. The second fabric cover layer 142 may be made of a low-friction material that is breathable. The low-friction material may provide an exterior surface that allows for low resistance during movement between the second pad member 140 and patient's skin. Comfort and support layers underlying the second fabric cover layer 142 are encased by the flexible and breathable material of the second fabric cover layer 142. The material of the second fabric cover layer 142 may be durable enough to hold the comfort and support layers in place at a fixed central area upon cover 125. The second fabric cover layer 142 may be comprised of materials such as cotton, polyester, synthetic rubber, neoprene, nylon, spandex, polyurethane, or a combination of the materials, separated or blended, to ensure a clean and comfortable application of the medical device 100 against the patient's skin.

The second pad member 140 comprises a second support layer 144 that underlies the second fabric cover layer 142 and is adjacent to cover 125. The second support layer 144 may be structured to support overlying layers of the second pad member 140. The second support layer 144 may be made of higher resilient, low compression set materials to provide support and resistance with pressure applied to the second pad member 140. The higher resilient material may be a high-density, less porous, less pliable, firm foam, sponge, rubber, or blended combination that may yield under higher degrees of compression. Foam utilized in the second support layer 144 as the higher resilient material may come from closed cell foam materials having high-density, such as foams from the family of polymers comprising polyethylene, polyurethane, silicone, and polyvinyl chloride (PVC). Sponge utilized in the second support layer 144 may comprise elastomers, like ethylene propylene diene terpolymer (EPDM) and Neoprene sponge having closed cells. Rubber utilized in the second support layer 144 may comprise elastomers that do not necessarily fit within the foam and sponge categories, like butyl rubber, cork, and cork rubber. A blended combination of these materials or other specialty foam materials having characteristics of low to non-sponginess may be utilized as the higher resilient material of the second support layer 144. The second support layer 144 has a greater density than a second comfort layer 146 of the second pad member 140.

The second comfort layer 146 may be disposed between the second fabric cover layer 142 and the second support layer 144. The second comfort layer 146 may be structured to provide cushioning between the patient's skin contacting the second fabric cover layer 142 and the higher resilient material of the second support layer 144. The second comfort layer 146 may be made of lower resilient, high compression set materials to prevent occlusion of intravenous vessels. The lower resilient material may be a low-density, more porous, more pliable, soft foam, sponge, rubber, or blended combination that may yield under lower degrees of compression. Foam utilized in the second comfort layer 146 as the lower resilient material may come from closed cell foam materials and open cell foam materials having low-density, such as foams from the family of polymers comprising urethane foam options, like ether or ester, microcellular urethane, memory foam, and reticulated filter foam. Sponge utilized in the second comfort layer 146 may comprise elastomers, like Neoprene sponge having open cells. Rubber utilized in the second comfort layer 146 may comprise elastomers that do not necessarily fit within the foam and sponge categories. A blended combination of these materials or other materials having greater sponginess may be utilized as the lower resilient material of the second comfort layer 146 to enhance patient comfort and reduce the risk of pressure and irritation at the intravenous catheterization therapy treatment site of the patient.

In FIG. 1, the first pad member 130 and the second pad member 140 are shown parallel and laterally spaced apart. Each of the dual pad members has a flat-bottomed capsule shape. Together, the dual pad members are shaped similar to a pair of flat-bottomed capsules that are laterally separated. Alternatively, a single pad member may be disposed on the base 120. The single pad member may be bifurcated. Alternatively, the single pad member may be square-shaped, triangle-shaped, or rectangular-shaped with a central opening or cavity disposed inward of the boundaries of the single pad member. The shape of the pad member(s) may conform to a contour in the bend of a patient's joint adjacent and about the intravenous catheterization therapy treatment site. The shape of the central opening may conform to the area of the to the intravenous catheterization therapy treatment site.

In the example given, the dual pad members are laterally spaced apart so as to define a central elongated opening 150. The central elongated opening 150 may mirror the intravenous catheterization therapy treatment site, positionally from above, when the cover 125 of the medical device 100 is in placement over the intravenous catheter inserted into a patient. The central elongated opening 150 may be adjacent to cover 125. The separation distance between the dual pad members defines the width of the central elongated opening 150. The width of the central elongated opening 150 may be between 1/32 inch to 1 inch wide. The width of the central elongated opening 150 may be further between 1/16 inch to ⅞ inch, between ¼ inch to 1 inch, between ¼ inch to ½ inch, between ½ inch to 1 inch, between ¼ inch to ¾ inch, between 1/16 inch to ¼ inch, between 1/32 inch to ¼ inch, between 1/32 inch to ⅛ inch, between 1/32 inch to 1/16 inch wide.

In the example of medical device 100 shown, the longitudinal length of each of the dual pad members may be between 1 inch and 4 inches. The length of each of the dual pad members may be further between 1¼ inches to 3⅞ inches, between 1½ inches to 3½ inches, between 1¼ inches to 3¼ inches, between 1¼ inches to 3 inches, between 1¼ inches to 3½ inches, between 1½ inches to 3 inches, between 1¼ inches to 2¾ inches, between 1 inch to 2 inches, between 1¼ inches to 2½ inches, between 1½ inches to 2 inches, between 1¼ inches to 1¾ inches, between 1 inch to 2 inches, between 1 inch to 2¼ inches, between 1 inch to 2⅛ inches, between 1 inch to 2 1/16 inches.

Each of the dual pad members extend outwardly from the base 120 so as to define a pad height 138, which as a result defines the depth of the central elongated opening 150. The pad height 138 along the longitudinal length of each of the dual pad members may be between 1/32 inch to 2 inches. The pad height 138 along the longitudinal length of each of the dual pad members may be further between ¼ inch to 1⅞ inches, between ½ inch to 1½ inches, between ¼ inch to 1¼ inches, between ¼ inch to 1 inch, between ¼ inch to ½ inch, between ½ inch to 1 inch, between ¼ inch to ¾ inch, between 1 inch to 2 inches, between 1¼ inches to 1½ inches, between 1½ inches to 2 inches, between 1¼ inches to 1¾ inches, between 1/32 inch to 1 inch, between 1/32 inch to ¼ inch, between 1/32 inch to ⅛ inch, between 1/32 inch to 1/16 inch.

The thickness of the comfort layer versus the support layer may be based on a fraction or ratio of the pad height 138. In one example, the thickness of the support layer is approx. ⅓ of the total pad height and the thickness of the comfort layer is approx. ⅔ of the total pad height. In another example, the thickness of the support layer is approx. ¼ of the total pad height and the thickness of the comfort layer is approx. ¾ of the total pad height. In another example, the thickness of the support layer is approx. ⅗ of the total pad height and the thickness of the comfort layer is approx. ⅖ of the total pad height. The thickness of the differing density layers of the pad members may be based on the size and age of the patient being treated.

The pad height 138 along the longitudinal length of each of the pad members may be lesser or greater in height depending on which longitudinal portion of the pad member is being referred to. A first longitudinal portion 152 of the pad member may have a first longitudinal portion end height. A second longitudinal portion 156 of the pad member may have a second longitudinal portion end height. A longitudinal center portion 154 of the pad member may have a longitudinal center portion height. In one example, the elongated pad member may be approximately 3 inches in length, where the first longitudinal portion 152 has a length of a ½ inch and a first longitudinal end height of a ½ inch, the longitudinal center portion 154 has a length of 1½ inches and a longitudinal center portion height of 1 inch, and the second longitudinal portion 156 has a length of a ½ inch and a second longitudinal portion end height of a ½ inch. In another example, the elongated pad member may be approximately 4 inches in length, where the first longitudinal portion 152 has a length of a 1 inch and a first longitudinal portion end height of a ¾ inch, the longitudinal center portion 154 has a length of 2 inches and a longitudinal center portion height of 1½ inches, and the second longitudinal portion 156 has a length of a 1 inch and a second longitudinal portion end height of a ¾ inch.

In the case where the exterior surface of the pad member is curved convex away from the cover 125 along the longitudinal length of the pad member, the pad height 138 of each of the first longitudinal portion 152 and second longitudinal portion 156 may transition with an increasing taper towards the longitudinal center portion 154 and transition with a decreasing taper towards the terminal ends where each of the pad members intersect with the cover 125. In such example, the longitudinal center portion height is greater between the cover 125 and the patient's skin than the first longitudinal portion end height and the second longitudinal portion end height between the cover 125 and the patient's skin. Tapering the pad height 138 longitudinally between the center portion and the end portions of the pad member may allow the pad member to conform to a bend or pocket of a joint of the patient, such as the antecubital fossa of a patient's elbow.

Additionally, the exterior surface of the pad member may be curved convex away from the cover 125 across a width of the single pad member from a left-hand side of the single pad member to a right-hand side of the single pad member to accommodate an intravenous catheter line. In an example of the medical device 100 that utilizes a single pad member disposed on the base 120, the single pad member may be concavely curved towards the cover 125 across a width of the single pad member from a left-hand side of the single pad member to a right-hand side of the single pad member to accommodate an intravenous catheter line.

Often, the antecubital fossa may be the first place a nurse might utilize to insert an intravenous catheter. Although the antecubital fossa provides a quick and easy site to establish intravenous therapy, maintaining patency of the treatment site over time with catheter line placement in the area of a joint may become problematic. Since joints bend, the catheter line may become occluded resulting in the decreased flow or stoppage of medication being administered. Additionally, every time a patient bends the joint, infiltration and irritation of the catheter line may occur which decreases the longevity and effectiveness of the intravenous catheterization therapy treatment site. More time from the nurse is demanded to perform intravenous catheter insertions, adjust administration of medications, which in turn may cause lowered patient satisfaction and medical errors.

As a result, designs to prevent a patient from articulating the joint, such as restraints applied to a portion of the patient to protect catheter lines, may be utilized for short-term medical immobilizations. Restraints utilized for medical immobilizations, especially the use of bulky or rigid materials in these securing devices, can cause discomfort to the patient, immobility of the patient's joint, pressure point sores, and physical and psychological trauma, particularly in long-term medical immobilizations. In one example, with intravenous catheterization therapy administered at an antecubital fossa treatment site, an intravenous arm board may be utilized to protect and maintain patency of the catheter line during the recovery process. Patients in restraints require frequent monitoring and assessments. In contrast, medical device 100 of this disclosure not only improves patency and longevity of the intravenous catheterization therapy treatment site with comfortable application, but also allows the patient substantive mobility of their joint and ease of assessment and dressing changes of the intravenous catheterization therapy treatment site.

FIG. 4 depicts a top perspective view of the medical device 100 secured around a joint 405 of a patient 10, such as an elbow joint, with the base 120 applied to the antecubital fossa area. Medical device 100 is structured to have adjustability while providing adequate support to prevent dislodgement or movement of an intravenous catheter 410 and/or intravenous cannula 400 during patient activity.

Medical device 100 incorporating the base 120 that slides upon the securement 110 facilitates positioning of the cover 125 above the intravenous catheterization therapy treatment site. Sliding the base 120 upon the securement 110 may move the first pad member 130 and second pad member 140 disposed upon the cover 125 into a treatment position above the intravenous catheterization therapy treatment site. Base 120 may be moved as shown by movement arrow 129 to position the first pad member 130 and second pad member 140 on either side of a longitudinal axis 450 of the intravenous catheter 410 inserted into patient 10.

Medical device 100 incorporating a cover 125 that swivels facilitates positioning of the dual pad members, when disposed below the securement 110, against the patient's skin at a treatment position that is above and adjacent the intravenous catheterization therapy treatment site. Swiveling the base 120 clockwise (movement arrow 126) or counterclockwise (movement arrow 128) upon the securement 110 aligns the central elongated opening 150, which is disposed between the first pad member 130 and second pad member 140, with the longitudinal axis 450 of the intravenous catheter 410.

Alignment of the central elongated opening 150 with the longitudinal axis 450, also aligns the respective longitudinal axes 135, 145 of the first pad member 130 and second pad member 140 so that they are laterally adjacent to and parallel with the longitudinal axis 450. Disposing the first pad member 130 and second pad member 140 laterally adjacent and parallel with the longitudinal axis 450 facilitates patency of the intravenous catheter 410 that is disposed adjacent and below the dual pad members. The dual pad members protect and stabilize the intravenous catheter 410 inserted into the patient 10 at the intravenous catheterization therapy treatment site.

Medical device 100 incorporating a securement 110 band with fastener attachments provides adjustable and comfortable securement of the medical device 100 to a portion of the patient 10 undergoing intravenous catheterization therapy. A pocket, being a patch of greater flexibility material than the securement 110 band, may be centrally located within the securement 110 band. The pocket may be sized to accommodate differing patient elbow sizes (i.e. small, medium, large, etc.)

As shown in FIG. 2, a first pocket 162 is disposed within the securement 110 at the first end 112. A second pocket 164 is disposed within the securement 110 at the second end 114. With securement 110 band secured in place around joint 405 of patient 10, the first pocket 162 and second pocket 164 may be disposed opposite the cover 125 to receive the apex of the elbow joint inherent in FIG. 4. Placement of the apex of the elbow joint (i.e. olecranon) within the pockets with securement 110 placed around the elbow promotes freedom in the range of motion by the patient. A first reinforcement 166 and a second reinforcement 168, being strips of less flexibility material than the first pocket 162 and second pocket 164, may be disposed across the first pocket 162 and second pocket 164, respectively, to provide resistance against the elbow joint received.

As shown in FIG. 4, with securement of the medical device 100 to the portion of the patient 10 undergoing intravenous therapy, at least a portion of the first pad member 130 and second pad member 140 are disposed directly below a portion of the securement 110 band. Swiveling of the cover 125 upon the securement 110 band may be resisted with the securement 110 band secured about the elbow of the patient 10. Medical device 100 is secured to the patient 10 and stabilizes patency of intravenous catheter 410 without having to be fastened, connected, or attached to the components of the intravenous cannula 400 by any means. The dual pad member displace pressure induced by securement of the securement 110 away from the intravenous catheter 410 so that occlusion of the intravenous catheter 410 does not occur even when the elbow is flexed.

The first pad member 130 and second pad member 140 are structured, as discussed above, to maintain the central elongated opening 150 when they are bent along respective longitudinal axes 135, 145. When the joint 405 upon which the medical device 100 is applied and secured bends, the first pad member 130 and second pad member 140 bridge the gap of the joint 405 to resist maximum flexion (in the case of an elbow joint) or maximum extension (in the case of a wrist joint) of the joint angle (a) by between 5-50%. In the case of an elbow joint, the amount of maximum flexion of the joint angle (a) that may be resisted may be further between 5-45%, between 5-40%, between 5-35%, between 5-30%, between 5-25%, between 5-20%, between 5-15%, or between 5-10%. The flexible design of the medical device allows for a substantive range of motion of the joint 405 while preventing occlusion of the intravenous catheter 410 accommodated by the central elongated opening 150.

The first pad member 130 and second pad member 140 displace pressure to either side of the intravenous catheter 410, without discomfort to the patient 10, when the joint 405 is flexed. To do so, the first pad member 130 and second pad member 140 utilize the combination of differing density layers, as discussed above, to displace pressure from the securement 110 through the pad members and adjacent tissues to avoid occlusion of the intravenous catheter 410 accommodated by the central elongated opening 150. The pad member(s) may be shaped to also accommodate a joint shape, as well as include a channel or cavity shaped to displace pressure away from the intravenous catheter 410. Medical device 100 does not necessarily have to be utilized at a joint area (i.e. elbow or wrist), but may be used on any portion (i.e. forearm) of the patient 10 to stabilize and maintain patency of the intravenous catheterization therapy treatment site.

A sleeve (not shown), that may be elastic, can be slid over the medical device 100 applied to the patient 10 to aid in comfort, aesthetics, and prevent any portion of the medical device 100 from being snagged. The sleeve may be separate and unattached to the medical device 100, or attached (i.e. sewn) to an edge of the securement 110.

Integration of the disclosed elements, such as the swivel function and padding design, results in a medical device that is optimized to provide support and comfort for the patient, while maintaining the effectiveness of the medical intervention being applied. The design of the medical device 100 is specifically tailored to address the challenges associated with positioning, securing, and stabilizing therapy supports of intravenous catheters. Medical device 100 combines the features described, such as adjustable securement, multi-density padding elements, and swiveling functionality, to provide a comprehensive solution that optimizes patient outcomes.

It is understood that the invention is not confined to the particular construction and arrangement of parts herein described. That although the drawings and specification set forth a preferred embodiment, and although specific terms are employed, they are used in a description sense only and embody all such forms as come within the scope of the following claims.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims.

For the convenience of the reader, the above description has focused on a representative sample of all possible embodiments, a sample that teaches the principles of the invention and conveys the best mode contemplated for carrying it out. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed variations, modifications and variations are within the literal scope of the following claims, and others are equivalent.

I claim:

1. A medical device comprising:
a securement band;
a cover structured to swivel upon the securement band;
a first pad member that is elongated and resilient extending outwardly away from the cover, the first pad member comprising:
i. a first fabric cover layer that is a low-friction material structured to contact the skin of a patient;
ii. a first support layer adjacent to the cover;
iii. a first comfort layer between the first fabric cover layer and the first support layer, wherein the first support layer has greater density than the first comfort layer;
a second pad member that is elongated and resilient extending outwardly away from the cover, the second pad member comprising:
i. a second fabric cover layer that is a low-friction material structured to contact the skin of the patient;
ii. a second support layer adjacent to the cover;
iii. a second comfort layer between the second fabric cover layer and the second support layer, wherein the second support layer has greater density than the second comfort layer;
wherein the first pad member and the second pad member are parallel and laterally spaced apart so as to define a central elongated opening adjacent to the cover and whereupon swiveling the cover upon the securement band positions the central elongated opening to facilitate an intravenous catheter disposed below and between the first pad member and the second pad member; and
at least a portion of the first pad member and the second pad member being disposed directly below a portion of the securement band when the securement band is secured to a portion of the patient stabilizes patency of the intravenous catheter.

2. The medical device of claim 1, the securement band comprising:
an elbow pocket that is a patch of greater flexibility material centrally located within the securement band and opposite the cover with the securement band secured to the portion of the patient.

3. The medical device of claim 1, wherein the securement band secured to the portion of the patient resists swiveling of the cover upon the securement band.

4. The medical device of claim 1, wherein the first pad member and the second pad member applied to the patient resist maximum flexion of a joint by between 5% to 50%.

5. The medical device of claim 1, wherein a height of the first pad member is between ¼ inch to 1 inch.

6. The medical device of claim 1, where the first pad member further comprises:
a first longitudinal portion having a first longitudinal portion end height;
a second longitudinal portion having a second longitudinal portion end height;
a longitudinal center portion having a longitudinal center portion height; and
wherein the longitudinal center portion height is greater between the cover and the skin of the patient than the first longitudinal portion end height and the second longitudinal portion end height.

7. The medical device of claim 1, wherein the first pad member and the second pad member are structured to maintain the central elongated opening when they are bent along their respective longitudinal axes when a joint upon which the medical device is secured bends so as to prevent occlusion of the intravenous catheter accommodated by the central elongated opening.

8. The medical device of claim 1, wherein the first support layer is a high-density foam and the first comfort layer is a low-density foam.

9. The medical device of claim 1, wherein the medical device is secured to the patient without being fastened to the intravenous catheter.

10. A medical device comprising:
a securement band;
a cover mounted to the securement band;
a first pad member extended outwardly away from the cover;
a second pad member extended outwardly away from the cover; and
wherein the first pad member and the second pad member are parallel and laterally spaced apart so as to define a central elongated opening adjacent to the cover to facilitate an intravenous catheter disposed below and between the first pad member and the second pad member.

11. The medical device of claim 10, wherein the cover is structured to swivel upon the securement band.

12. The medical device of claim 10, wherein the first pad member comprises:
a first fabric cover layer that is a low-friction material structured to contact the skin of a patient.

13. The medical device of claim 12, wherein the first pad member further comprises:
a first comfort layer disposed between the first fabric cover layer and a first support layer disposed adjacent to the cover.

14. The medical device of claim 13, wherein the first comfort layer has a lower density than the first support layer.

15. The medical device of claim 10, where the first pad member comprises:
a first longitudinal portion having a first longitudinal portion end height;
a second longitudinal portion having a second longitudinal portion end height;
a longitudinal center portion having a longitudinal center portion height; and
wherein the longitudinal center portion height of the first pad member extended outwardly away from the cover is greater than the first longitudinal portion end height and the second longitudinal portion end height.

16. A medical device comprising:
a securement band;
a cover structured to swivel upon the securement band;
a first pad member extended outwardly away from the cover;
a second pad member extended outwardly away from the cover, wherein the first pad member and the second pad member are parallel and laterally spaced apart so as to define a central elongated opening adjacent to the cover; and
whereupon swiveling the cover upon the securement band positions the central elongated opening to facilitate an intravenous catheter disposed below and between the first pad member and the second pad member.

17. The medical device of claim 16, wherein the securement band secured to the patient resists swiveling of the cover upon the securement band.

18. The medical device of claim 16, further comprising:
a swivel mechanism disposed between the securement band and the cover to swivel the cover upon the securement band.

19. The medical device of claim 16, wherein a range of rotation of the cover upon the securement band is between 5 degrees to 360 degrees.

20. The medical device of claim 16, wherein alignment of the central elongated opening with a longitudinal axis of the intravenous catheter stabilizes patency of the intravenous catheter to prevent occlusion of the intravenous catheter with the securement band secured to the patient.
